(19) United States
(12) Patent Application Publication
(10) Pub. No.: US 2011/0166560 A1
(43) Pub. Date: Jul. 7, 2011

(54) SKIN CARE LASER DEVICE

(75) Inventor: I-Ku Kuo, Thaipao City (TW)

(73) Assignee: SOLAR SYSTEM BEAUTY CORPORATION, Thaipao City (TW)

(21) Appl. No.: 12/683,782

(22) Filed: Jan. 7, 2010

(51) Int. Cl. A61B 18/20; H04N 5/335; H04N 5/33

(52) U.S. Cl. 606/9; 348/308; 348/164; 348/E05.09; 348/E05.091

(57) ABSTRACT

A skin care laser device is provided, which mainly includes a photograph unit, a distance measuring unit, a computing unit, a laser unit, a moving unit, and a comparing unit. The photograph unit scans a patient and generates an image signal. The distance measuring unit measures a distance from the patient and generates a distance signal. The computing unit receives the image signal and the distance signal, computes the signals, and generates a displacement instruction and a control instruction. The moving unit receives the displacement instruction and drives the photograph unit, the distance measuring unit, and the laser unit to make dynamic movement. The comparing unit receives the control instruction, compares an energy data of the laser unit with a control data of the control instruction, and determines whether to excite a laser to the patient or not.

### BACKGROUND OF THE INVENTION

1. Field of Invention

The present invention relates to a skin care laser device, and more particularly to a skin care laser device that can scan images of a target surface of a patient in advance, recognize areas under treatment, and measure a distance between each treatment point and a laser beam source, such that the laser beam source automatically adjusts energy intensities for treating different treatment points according to raised and depressed areas under treatment.

2. Related Art

The invention of laser in the year of 1960 represents a breakthrough in the field of science and technology. As a laser beam source can emit light beams with ultra-high energy intensities within a very short time and produce selective damages to tissues, the laser has been widely applied in clinical medicine.

Nowadays, the technology of applying the laser in the field of medical cosmetology becomes one of the most popular technologies, which includes various types of techniques and has been applied in a wide treatment range. However, the corresponding treatment modes are more or less the same. Currently, the common technologies mainly utilize robot arms and light guide fibers. In Taiwan Patent No. 1283593 entitled “AUTOMATIC LASER DISPLACEMENT CONTROL METHOD OF SKIN LASER TREATMENT EQUIPMENT”, laser beams are guided to each treatment point according to a coordinate position of an area under treatment through an image positioning control mechanism, so as to achieve an automatic operation process.

In the above technology, an operator is enabled to carry out the treatment without manually holding the instruments, which not only avoids the mistakes made due to manually holding the instruments, but also ensures the precise control on the area under treatment. Furthermore, through the image positioning control mechanism, the energy of the laser beams for treatment is stable and uniform. Meanwhile, the operator for carrying out the treatment does not need to directly stare at the treatment positions, thereby protecting the eyes from being hurt due to the intense laser beams. Although the prior invention has already achieved such advanced effects, great challenges still exist in processing raised and depressed areas under treatment. Because of the raised and depressed areas under treatment, the distances between the treatment points and the laser beam source are varied. If the laser beams with the same intensity emitted by the laser beam source are applied, the achieved effects may be somewhat deviated from the originally expected effects. Therefore, the technology can be further improved.

### SUMMARY OF THE INVENTION

In view of the above, the present invention is directed to a skin care laser device, which is applicable to scan images of a surface of a patient in advance, to recognize areas under treatment, and to measure a distance between each treatment point and a laser beam source, such that the laser beam source automatically adjusts energy intensities for treating different treatment points according to raised and depressed areas under treatment.

The present invention provides a skin care laser device, which includes a photograph unit, a distance measuring unit, a computing unit, a laser unit, a moving unit, and a comparing unit. The photograph unit scans a patient and generates an image signal. The distance measuring unit measures a distance from the patient and generates a distance signal. The computing unit receives the image signal and the distance signal, computes the signals, and generates a displacement instruction and a control instruction. The moving unit receives the displacement instruction and drives the photograph unit, the distance measuring unit, and the laser unit to make dynamic movements. The comparing unit receives the control instruction, compares an energy data of the laser unit with a control data of the control instruction, and determines whether to excite a laser to the patient or not.

As compared with a structure in the prior art, in the skin care laser device of the present invention, the distance measuring unit is added to record raised and depressed areas of the patient, and the moving unit is added to control the photograph unit, the distance measuring unit, and the laser unit to make tri-axial displacement, so as to cater to the raised and depressed treatment positions at different areas of the patient. In such a manner, a cosmetic effect expected by the operator or even expected by the patient can be realized as much as possible.

### BRIEF DESCRIPTION OF THE DRAWINGS

FIG. 1 is a structural diagram of a skin care laser device according to the present invention; and

FIG. 2 is a flow chart of operations of a skin care laser device according to the present invention.

### DETAILED DESCRIPTION OF THE INVENTION

A skin care laser device according to a preferred embodiment of the present invention is illustrated below with reference to the accompanying drawings, and the same reference numerals represent the same elements.

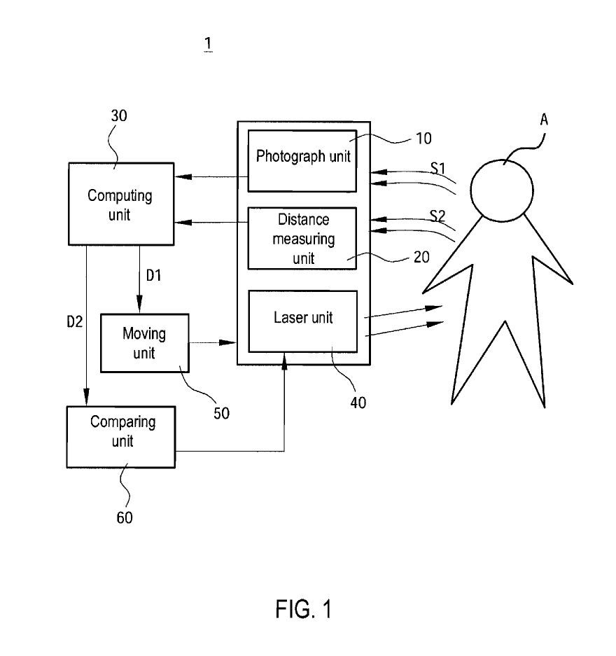

FIG. 1 is a structural diagram of a skin care laser device according to the present invention. Referring to FIG. 1, a skin care laser device 1 includes a photograph unit 10, a distance measuring unit 20, a computing unit 30, a laser unit 40, a moving unit 50, and a comparing unit 60. The photograph unit 10 scans a patient A and generates an image signal S1. The distance measuring unit 20 measures a distance from the patient A and generates a distance signal S2. The computing unit 30 receives the image signal S1 and the distance signal S2, computes the signals S1 and S2, and generates a displacement instruction D1 and a control instruction D2. The moving unit 50 receives the displacement instruction D1 and drives the photograph unit 10, the distance measuring unit 20, and the laser unit 40 to make dynamic movements. The comparing unit 60 receives the control instruction D2, compares an energy data of the laser unit 40 with a control data of the control instruction D2, and determines whether to actuate the laser unit 40 to excite a laser to the patient A or not.

As the skin care laser device 1 includes the comparing unit 60, it may be considered that a prescribed dose of energy is confirmed once again before the laser is excited. In the situations that the dose of energy is too high or too low, the comparing unit 60 may send an interrupt signal to the laser unit 40 to ensure the safety of the patient A.

In this embodiment, the photograph unit 10 adopts a charge coupled device (CCD) lens or a complementary metal oxide semiconductor (CMOS) lens. The distance measuring unit 20 is implemented as an ultrasonic sensor or an infrared sensor. Moreover, the computing unit 30 is implemented as a computer with a display, in which the display is used for displaying the image signal and various data corresponding to the displacement instruction D1 and the control instruction D2. The display even may be a touch screen, such that the operator can directly adjust data and control an operation of the computing unit 30 on the screen. In addition, the laser unit 40 further includes a transmission device therein, in which the transmission device is used for guiding and outputting the laser and the transmission device here may be an optical fiber or optical lens module.

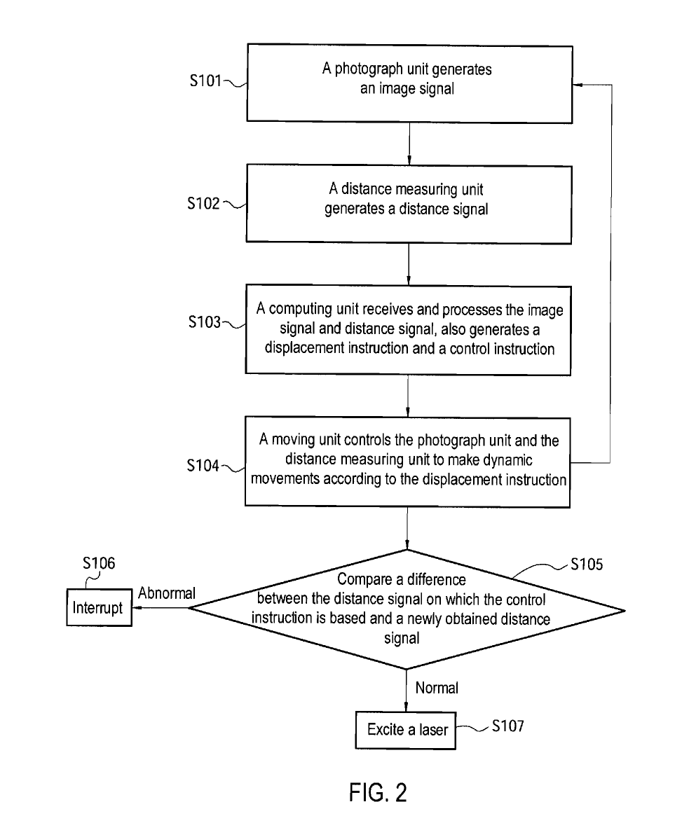

Next, FIG. 2 is a flow chart of an operation of a skin care laser device according to the present invention. Referring to FIGS. 1 and 2 at the same time, in Step S101, the photograph unit 10 in the skin care laser device 1 first scans and records a profile of a patient A and accordingly generates an image signal S1. Meanwhile, in Step S102, the distance measuring unit 20 measures a distance from the patient A and accordingly generates a distance signal S2. In Step S103, the computing unit 30 receives the image signal S1 and the distance signal S2, computes the signals through pre-stored programs and stores the computed signals in a database, and generates a displacement instruction D1 and a control instruction D2. In Step S104, the displacement instruction D1 first instructs the moving unit 50 to control the photograph unit 10, the distance measuring unit 20, and the laser unit 40 to make movements in a tri-axial coordinate space, such that the units 10, 20, and 40 may adjust their coordinates at any time. At this time, after Steps S101 to S104 are performed once again, the distance signal S2 returned by the distance measuring unit 20 is computed by the computing unit 30 again, which is then compared with the applied dose of energy in the original control instruction D2 in Step S105. If the dose of energy is too high or too low, in Step S106, an interrupt signal is output to the laser unit 40 to stop the excitation temporarily. At this time, the computing unit 30 once again gives a control instruction D2 containing a dose indication, so as to apply a laser to the area under treatment again. If the dose of energy to be applied is the same as the originally set dose of energy or the difference there-between is tolerable, in Step S107, the laser unit 40 is controlled to excite a laser to the patient A.

In conclusion, in the skin care laser device according to the present invention, the distance measuring unit is added to record the raised and depressed areas of the patient, and the moving unit controls the photograph unit, the distance measuring unit, and the laser unit to make tri-axial movements, so as to cater to treatment positions at different heights, thereby approaching the practically needed functions and greatly enhancing the quality of the medical treatment effect. Moreover, in the present invention, due to the comparing unit, the dose of energy for laser excitation can be confirmed again before the laser is applied, which can be considered as another protection measure, thereby further ensuring the treatment safety for the patient.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A skin care laser device, comprising:
a photograph unit, for scanning a patient and generating an image signal;
a distance measuring unit, for measuring a distance from the patient and generating a distance signal;
a computing unit, for receiving the image signal and the distance signal, computing the signals, and generating a displacement instruction and a control instruction;
a laser unit;
a moving unit, for receiving the displacement instruction and driving the photograph unit, the distance measuring unit, and the laser unit to make dynamic movements; and
a comparing unit, for receiving the control instruction, comparing an energy data of the laser unit with a control data of the control instruction, and determining whether to actuate the laser unit or not.

2. The skin care laser device according to claim 1, wherein the comparing unit generates an interrupt signal to stop an operation of the laser unit when the energy data is different from the control data.

3. The skin care laser device according to claim 1, wherein the photograph unit comprises a charge coupled device (CCD) lens or a complementary metal oxide semiconductor (CMOS) lens.

4. The skin care laser device according to claim 1, wherein the distance measuring unit is an ultrasonic sensor or an infrared sensor.

5. The skin care laser device according to claim 1, wherein the computing unit further comprises a display for displaying the image signal and data corresponding to the displacement instruction and the control instruction.

6. The skin care laser device according to claim 1, wherein the laser unit further comprises a transmission device for guiding and outputting a laser.

7. The skin care laser device according to claim 6, wherein the transmission device is an optical fiber or optical lens module.
